(19) United States
(12) Patent Application Publication
(10) Pub. No.: US 2008/0262082 A1
(43) Pub. Date: Oct. 23, 2008

(54) COMPOSITION CONTAINING S-(-)-TULIPALIN B OR ACETYLATED-S-(-)-TULIPALIN B

(75) Inventors: Deok Hoon PARK, Yongin-si (KR); Ji Young LEE, Gunpo-si (KR); Jongsung LEE, Ahnyang-si (KR)

Correspondence Address: ROTHWELL, FIGG, ERNST & MANBECK, P.C., 1425 K STREET, N.W., SUITE 800, WASHINGTON, DC, 20005, US

(73) Assignee: BIO SPECTRUM, INC., Gunpo-si (KR)

(21) Appl. No.: 12/105,757

(22) Filed: Apr. 18, 2008

(30) Foreign Application Priority Data
Apr. 20, 2007 (KR) 10-2007-0039050

(51) Int. Cl. A61K 31/341; C07D 307/20; A61P 31/04

(52) U.S. Cl. 514/473; 549/477

(57) ABSTRACT

The present invention provides S-(−)-tulipalin B, acetylated-S-(−)-tulipalin B and an antibacterial composition comprising the same. The active compound tulipalin B and composition of the present invention exhibit superior and stable antibacterial activity over a broad range of temperature and pH, as compared to conventional natural antibacterial agents used in related industries. Therefore, the composition and active compound of the present invention can be widely used as a natural food preservative and/or an antibacterial agent in a variety of fields such as foods, pharmaceuticals, cosmetics, fibers, livingwares, and the like.

### RELATED APPLICATIONS

The present disclosure relates to subject matter contained in priority Korean Application No. 10-2007-0039050, filed on Apr. 20, 2007, which is herein expressly incorporated by reference in its entirety.

### DESCRIPTION OF THE DRAWINGS

FIG. 1 illustrates a flow process chart for preparation of S-(−)-tulipalin B according to the present invention.

### DESCRIPTION OF THE INVENTION

### Technical Field and Background Art

The present invention relates to S-(−)-tulipalin B or acetylated-S-(−)-tulipalin B represented by formula 1:

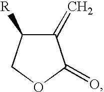

and an antibacterial composition comprising the same.

As is well known, foodstuffs, cosmetics, fibers, and pharmaceuticals inevitably require preservatives against microbial decay in order to maintain product quality for a long period of time. This is particularly true of almost all cosmetic preparations which have a relatively long circulation period and are rich in nutrients necessary for growth and proliferation of microorganisms.

Paraben preservatives, which have been conventionally and widely used in cosmetic, food and pharmaceutical industries, suffer from various potential problems such as skin allergy and emergence of resistant bacteria. In order to overcome these disadvantages, a great deal of research and study continues to focus on development of natural preservatives which are capable of achieving excellent safety and economic efficiency.

Most of natural antibacterial substances known hitherto suffer from various problems associated with undesirable colors and odors, poor stability, narrow antibacterial spectrum, and formulation problems, which in turn lead to difficulty in commercialization of antibacterial candidate drugs. Out of the natural antibacterial materials reported, only some of herbal extracts, such as Hinokitiol (Chamaecyparis obtusa extract), Magnolol (Magnolia officinalis extract), and DF-100 (grapefruit seed extract), have been commercialized in the market. In DF-100 which is far ahead of other counterparts in terms of product development and commercialization, its antibacterial activity is known to be due to organic acids and a synthetic preservative benzethonium chloride contained in DF-100. Other natural antibacterial agents still suffer from shortcomings such as economic infeasibility, narrow antibacterial spectrum, and poor physical properties resulting in limited applicability thereof. Therefore, there is an urgent need for natural antibacterial agents that have improved properties and that can be commercialized or incorporated into commercial products.

### Technical Problem

As a result of a variety of extensive and intensive studies and experiments to solve the problems as described above and to develop a natural antibacterial agent having improved properties, the inventors of the present invention investigated antibacterial and antifungal activity of about 200 Jeju island native plants and confirmed that S-(−)-tulipalin B from the leaf extract of Spiraea thunbergii Sieb has antibacterial effects against a broad spectrum of bacteria. The present invention has been completed based on these findings.

Therefore, the present invention has been made in view of the above problems, and it is an object of the present invention to provide a natural antibacterial agent which is capable of preventing deterioration or putrefaction of foods, pharmaceuticals or cosmetics occurring during storage or is capable of improving storability of such products, via use of an antibacterial ingredient S-(−)-tulipalin β isolated and purified from leaves of Spiraea thunbergii Sieb.

### Technical Solution

In accordance with an aspect of the present invention, the above and other objects can be accomplished by the provision of an antibacterial composition comprising S-(−)-tulipalin B or acetylated-S-(−)-tulipalin B represented by formula 1:

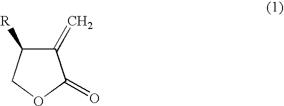

wherein R is hydroxy (—OH) or acetoxy (—OCOCH₃).

In the present invention, S-(−)-tulipalin B is a compound wherein R in formula 1 is hydroxy, and is referred to as (4S)-4-hydroxy-3-methylene-oxolan-2-one according to chemical (IUPAC) nomenclature. On the other hand, acetylated-S-(−)-tulipalin B is a compound wherein R in formula 1 is acetoxy and is referred to as (4S)-4-acetyl-3-methylene-oxolan-2-one.

S-(−)-tulipalin B or an acetylated version thereof may be extracted from plants containing such compounds, for example from leaves of Spiraea thunbergii Sieb. Extraction of active compounds may be carried out by a conventional method known in the art. Further, S-(−)-tulipalin B of the present invention may be prepared according to a conventional organic synthetic process known in the art.

More specifically, S-(−)-tulipalin B of the present invention is extracted as follows. First, a plant containing S-(−)-tulipalin B is ground into powder, and the powder is then mixed and stirred with ethanol to give an extract. The extract is concentrated to dryness under reduced pressure and ethyl acetate is added thereto to separate soluble components, followed by concentration to dryness. The aforesaid sample is dissolved in chloroform/methanol and is then adsorbed to a silica gel column. The adsorbed material is eluted at a very low flow rate with a varying volume ratio of chloroform:methanol, thus resulting in fractionation in constant volume aliquots. Among the fraction samples thus obtained, the fractions exhibiting strong inhibitory activity against E. coli are pooled and adsorbed once more to a silica gel column. The adsorbed material is re-eluted and fractionated with a mixed solvent of chloroform and methanol. Thereafter, among the fraction samples thus obtained, the fractions exhibiting strong inhibitory activity against E. coli are pooled and adsorbed once more to a C-18 flash column. The adsorbed material is then eluted at a high flow rate with a varying volume ratio of distilled water:methanol, whereby the fractionation is done in constant volume aliquots. Finally, the fractions exhibiting strong inhibitory activity against E. coli are pooled and purified by C-18 high-performance liquid chromatography (HPLC) to afford S-(−)-tulipalin B which is sought by the present invention.

The antibacterial composition of the present invention is effective for prevention or inhibition of bacterial infection of products. Examples of pathogenic bacteria that can be inhibited by the method of the present invention may include Streptococcus spp. such as Streptococcus pneumoniae, Streptococcus pyogenes, Streptococcus sanguis, Streptococcus sobrinus and Streptococcus mutans; Salmonella spp. such as Salmonella enteritidis and Salmonella typhimurium; Escherichia spp. such as Escherichia coli; Staphylococcus spp. such as Staphlylococcus aureus, Staphlylococcus epidermidis and Staphylococcus pneumoniae; Shigella spp. such as Shigella sonnei and Shigella flexneri; Pseudomonas spp. such as Pseudonmonas aeruginosa; Listeria spp. such as Listeria monocytogenes; Vibrio spp. such as Vibrio cholerae, Vibrio parahaemolyticuis, Vibrio anguillarnim, Vibrio splendidus and Vibrio vulnificus; Bacillis spp. such as Bacillus anthracis, Bacillis cereus and Bacillus subtilis; Propionibacterium spp. such as Propionibacterium acnes and Propionibacterium granulosum; Campylobacter spp. such as Campylobacter jejuni; Helicobacter spp. such as Helicobacter pylori; Clostridia spp. such as Clostridium difficile and Clostridium perfringens; Bordetella spp. such as Bordetella bronchiseptica; Yersinia spp. such as Yersinia enterocolitica; Legionella spp. such as Legionella pneumophila; Aeromonas spp. such as Aeromonas salmonicida; Bacteroides spp. such as Bacteroides fragilis; Neisseria spp. such as Neisseria meningitidis; Moraxella spp. such as Moraxella catarrhalis; Corynebacterium spp. such as Corynebacterium glutamicum; and Actinobacillis spp. such as Actinobacillus ureae.

The composition of the present invention may be used as a preservative for pharmaceuticals, foods, cosmetics, livingwares, and fibers. A content of the composition used as a preservative may be in a range of about 0.001% to about 30%, preferably about 0.001% to about 5%, more preferably about 0.001% to about 1% and the most preferably about 0.001% to about 0.02%. by weight based on the total weight of the preparation, even though it may vary depending upon various factors including characteristics and properties of the products to which the composition of the present invention will be applied.

When the composition of the present invention is employed in pharmaceutical preparations, the composition may be used in combination with optional additives that have been conventionally used in pharmaceutical chemistry, such as excipients, dispersants, disintegrants, sweeteners, glidants, fragrances, etc. Examples of the pharmaceutical preparation may include lotions, creams, injections, tablets and patches.

When it is desired to employ the composition of the present invention in foods, the composition may be used in combination with conventional ingredients that are known to those skilled in the art and are typically added to foods.

For cosmetic formulations, the composition may be combined with optional additives that have been conventionally used in the cosmetic industry, such as purified water, oils, surfactants, humectants, higher alcohols, chelating agents, pigments, fatty acids, antioxidants, preservatives, waxes, pH-adjusting agents, fragrances, etc. The composition of the present invention may be used in various forms of cosmetic formulations, such as lotions, essences, creams, skin lotions, and the like. Other ingredients used in individual formulations refer to materials that have been conventionally used in the cosmetic industry.

Further, the composition of the present invention may be used as a preservative for livingwares (such as soaps, hair shampoos, body shampoos, detergents, etc) and fibers.

The composition of the present invention may comprise an antioxidant to improve the stability of S-(−)-tulipalin B. Examples of the antioxidant may include Guaiac gum, cysteine hydrochloride, butylated hydroxytoluene (BHT), nordihydroguajaret acid, butylhydroxyanisol (BHA), and propyl gallate. Preferred is BHT. A content of the antioxidant may vary depending upon forms and intended applications of products in which the composition of the present invention is used. For example, the content of the antioxidant may be in a range of preferably about 0.0001 to about 0.01 ppm, and most preferably about 0.001 ppm.

### Mode for Invention

### EXAMPLES

Now, the present invention will be described in more detail with reference to the following Examples, Preparative Examples and Experimental Examples of S-(−)-tulipalin B and acetylated-S-(−)-tulipalin B. These examples are provided only for illustrating the present invention and should not be construed as limiting the scope and spirit of the present invention.

### Preparative Example 1

### Preparation of S-(−)-tulipalin B

1-1. Preparation of Raw Materials

Spiraea plant used as a source of raw materials was Spiraea thunbergii Sieb which may be domestically harvested from a plant habitat in Korea or is commercially available in the market. For easy and convenient extraction, the harvested plant was finely cut following drying at room temperature, or the dried sample was finely cut. Finely cutting (grinding) of the plant material was carried out by a conventional method, using a grinder mill.

1-2. Extraction of Active Ingredients

This step was intended for ethanol extraction of the dissolved solids from the plant material prepared in Section 1-1. Active ingredients were extracted from the ground sample of the previous step. As a natural antibacterial agent of the present invention was intended to be used for foods and cosmetics, fermentation ethanol (brewery grade) was selected as a non-toxic solvent for extraction of active ingredients. The plant sample and ethanol were mixed in a ratio of 2:1 (w/w), followed by extraction at room temperature for 2 weeks. More preferably, in order to increase the extraction efficiency of active ingredients, the extraction process was carried out for 1 week, followed by filtration. After the filtrate was separately concentrated, a two-fold volume of fermentation ethanol was added to the residue, followed by extraction for another 1 week. The filtrate thus obtained was completely evaporated in a rotary evaporator (EYILA, Japan) at 40° C. for 1 to 72 hours to thereby concentrate the extract. The concentrated plant extracts were ground into finely divided powder or were dissolved to a concentration of 100 mg/mL in ethanol and stored in a refrigerator until an antibacterial test.

1-3. Separation and Purification of Active Ingredients

To the dried sample was added a mixture of distilled water and ethyl acetate in a volume ratio of 1:2. Ingredients which are soluble in ethyl acetate were taken and concentrated to dryness.

The resulting concentrate sample of ethyl acetate-soluble ingredients was dissolved in 10 mL of a mixed solution of chloroform:methanol (CHCl₃:MeOH) (10:0) and then adsorbed to a silica gel column (70×400 mm). The adsorbed material was eluted at a slow flow rate of 0.5 mL/min using chloroform:methanol (gradient of from 98:2 to 93:7, 90:10 and 85:15), whereby the fractionation was done in constant volume aliquots of 20 mL.

The fractions exhibiting strong inhibitory activity against food-poisoning bacteria such as E. coli were pooled and were then adsorbed once more to a silica gel column (30×310 mm). The adsorbed material was re-eluted and fractionated with a mixed solvent of chloroform (CHCl₃):methanol (MeOH) (gradient of from 100:0 to 85:15 with a gradual increase of MeOH).

Out of the fractions thus eluted, the fractions exhibiting strong inhibitory activity against pathogenic bacteria such as E. coli were pooled and were then adsorbed to a C-18 reverse-phase chromatography column (adsorbent: 200 g, Analytichem BONDESIL C18, 40 μm, preparative grade, Varian, glass column; 10 mm i.d., 140 mm length). The adsorbed material was eluted with 500 mL of distilled water, followed sequentially by 1000 mL of 20% methanol and 40% methanol, and finally 1000 mL of 100% methanol.

Thereafter, the fractions exhibiting strong inhibitory activity against E. coli were pooled and purified by C-18 HPLC with distilled water:acetonitrile (H₂O:ACN) (gradient of from 100:0 to 94:6).

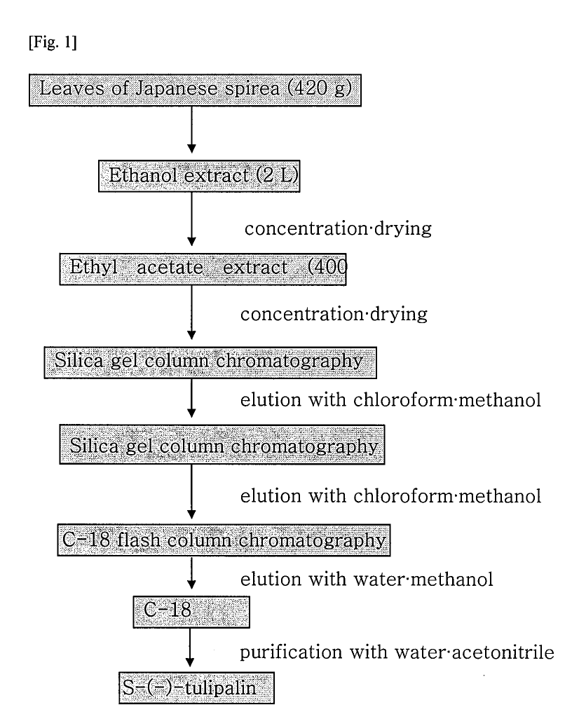

The resulting eluate was concentrated to dryness in a rotary vacuum evaporator at 40° C. to give about 46 mg of the title compound as an oil, which was sought by the present invention. FIG. 1 illustrates a process for extraction of S-(−)-tulipalin B. The analysis data for S-(−)-tulipalin B are given as follows.

[α]_{D}−0.11 (c=−4.632, MeOH); R_{f} 0.41 (Et₂O); UV λmax (H₂O) nm (log ε) 204 (3.96), EI MS m/z: 114 (C₅H₆O₃), ¹H NMR (300 MHz, D₂O) δ 6.52 (1H, d, J=1.5 Hz, H-3a), 6.23 (1H, d, J=1.5 Hz, H-3b), 5.10 (11H, ddd, J=1.5, 3.0, and 6.3 Hz, H-4), 4.68 (11H, dd, J=6.3 and 10 Hz, H-5a), 4.32 (11H, dd, J=3.0 and 10 Hz, H-5b) ¹³C NMR (100 MHz, D₂O) δ 172.8 (C-1), 137.2 (C-2), 128.8 (C-3), 67.2 (C-4), and 74.9 (C-5)

### Preparative Example 2

### Preparation of acetylated-S-(−)-tulipalin B

0.5 mL of acetic anhydride and 2 mL of triethylamine were added to 2 mL of an acetonitrile solution in which 10 mg of S-(−)-tulipalin B was dissolved, and the solution was stirred for 24 hours. A reaction mixture obtained after evaporation of the solution was distributed in 1.5 mL of methylene chloride and distilled water (2×2 mL), and an aqueous layer was taken and purified by C-18 HPLC with distilled water:acetonitrile (H₂O:ACN) (gradient of from 100:0 to 80:20) to thereby afford 8 mg of the title compound.

### Comparative Example 1

### Preparation of R-(+)-tulipalin B

In order to compare with antibacterial activity of S-(−)-tulipalin B, R-(+)-tulipalin B was isolated and purified from roots of tulip (Tulipa gesneriana) The bulb of tulip, from which isolation of R-(+)-tulipalin B was reported (J. C. M. Beijersbergen, 1972), was dried in the shade and mixed with methylene chloride in a ratio of 2:1 (w/w), followed by extraction at room temperature for 2 to 3 days. The extract was filtered and completely evaporated to concentration in a rotary evaporator (EYILA, Japan) at 40° C. for 1 to 72 hours.

The concentrated extract was dissolved in 10 mL of ethyl acetate:methanol (EtOAc:MeOH) (10:0) and was then adsorbed to a silica gel column (70×400 mm). The adsorbed material was eluted at a slow flow rate of 0.5 mL/min with ethyl acetate:methanol (gradient of from 100:0 to 80:20, 60:40, 40:20, 20:80, and 0:100), whereby the fractionation was done in constant volume aliquots of 20 mL.

Out of the eluted fractions, only the fractions containing R-(+)-tulipalin B were pooled, concentrated, and adsorbed to a C-18 reverse-phase chromatography column (adsorbent: 200 g, Analytichem BONDESIL C18, 40 μm, preparative grade, Varian, glass column; 10 mm i.d., 140 mm length). The adsorbed material was sequentially eluted with 1000 mL of 20% methanol and 40% methanol, and finally eluted with 1000 mL of 100% methanol.

The fractions, which were eluted with 20% methanol, were pooled and purified by C-18 HPLC with distilled water:acetonitrile (H₂O:ACN) (gradient of from 90:10 to 75:25).

The eluate was concentrated to dryness in a rotary vacuum evaporator at 40° C. to afford about 30 mg of the title compound R-(+)-tulipalin B which was sought by the present invention. This compound was designated (4R)-4-hydroxy-3-methylene-oxolan-2-one) according to IUPAC nomenclature.

### Example 1

### Lotion containing S-(−)-tulipalin B

A lotion containing S-(−)-tulipalin B was prepared according to the composition formula set forth in Table 1 below, using a conventional method known in the art.

TABLE 1

| Ingredients | Contents |
|---|---|
| S-(−)-tulipalin B | 1.00 |
| Ceteareth-6 olivate | 2.00 |
| Arachidyl alcohol/behenyl alcohol/arachidyl glucoside | 0.90 |
| Tocopheryl acetate | 0.05 |
| Neopentyl glycol diheptanoate | 2.00 |
| Polydecene | 2.50 |
| Stearic acid | 0.60 |
| Dimethicone | 0.40 |
| Triethanolamine | 0.10 |
| Disodium EDTA | 0.30 |
| Carbomer | 0.40 |
| Xanthan gum | 0.04 |
| Fragrance | Trace |
| Purified water | Balanced |
| Total | 100 |

### Experimental Example 1

### Antibacterial effects of S-(−)-tulipalin B and acetylated-S-(−)-tulipalin B on Pathogenic Bacteria (Aerobic)

In order to examine antibacterial activity of S-(−)-tulipalin B, acetylated-S-(−)-tulipalin B and R-(+)-tulipalin B, an antibacterial test was conducted for 18 species of pathogenic bacteria (aerobic) (5 Gram-positive species: Streptococcus mutans KCTC 3065, Staphylococcus aureus KCTC 1916, Pseudomonas aeruginosa KCTC 1637, Listeria monocytogenes KCTC 3569, and Bacillus subtilis KCTC 1021; and 13 Gram-negative species: Escherichia coli KCTC 1039, Salmonella typhimurium KCTC 2514, Salmonella enteritidis KCTC 12400, Shigella flexneri KCTC 2517, Shigella sonnei KCTC 2518, Vibrio parahaemolyticus KCTC 27, Bordetella bronchiseptica KCCM 21998, Yersinia enterocolitica KCCM 41657, Legionella pneumophila KCCM 41779, Aeromonas salmonicida KCTC 12266, Neisseria meningitidis KCCM 41562, Moraxella catarrhalis KCCM 40056, and Actiinobacillus ureae KCTC 2674). The antibacterial assay was carried out using an agar diffusion method. This method was based on determination of an antibiotic concentration in solutions of interest by applying a known concentration of an antibiotic solution to several discs, placing the antibiotic-treated discs on bacteria-seeded agar plates, culturing the bacteria, and measuring a diameter of a clear zone (growth inhibition halo) surrounding the antibiotic discs. The Kirby-Bauer method is currently widely used as an agar diffusion method. As a result of the antibacterial assay, Table 2 below shows that S-(−)-tulipalin B and acetylated-S-(−)-tulipalin B exhibited high antibacterial effects on all kinds of test bacteria, whereas R-(+)-tulipalin B exhibited no antibacterial effects on all kinds of bacteria even at a treatment concentration of 1.0%.

TABLE 2
Antibacterial effects of tulipalin B on aerobic bacteria
Antibacterial effects of tulipalin B on aerobic bacteria

| | | Treatment concentration (%)/Inhibitory zone diameter | | | | | | | | | | | | | | |
|---|---|---|---|---|---|---|---|---|---|---|---|---|---|---|---|---|
| | | (mm) | | | | | | | | | | | | | | |
| | | 1.0 | | | 0.3 | | | 0.1 | | | 0.03 | | | 0.01 | | |
| | | A* | B | C | A | B | C | A | B | C | A | B | C | A | B | C |
| Gram | S. mutans | 16 | 16 | — | 13 | 15 | — | 11 | 13 | — | — | 11 | — | — | — | — |
| (+) | S. aureus | 18 | 18 | — | 15 | 17 | — | 12 | 13 | — | — | 12 | — | — | — | — |
| | P. aeruginosa | 14 | 14 | — | 12 | 12 | — | — | 11 | — | — | — | — | — | — | — |
| | L. monocytogenes | 16 | 16 | — | 13 | 14 | — | 11 | 11 | — | — | 11 | — | — | — | — |
| | B. subtilis | 17 | 18 | — | 15 | 16 | — | 13 | 15 | — | 11 | 12 | — | — | — | — |
| Gram | E. coli | 20 | 20 | — | 16 | 18 | — | 13 | 16 | — | 11.5 | 13 | — | 10.5 | 11.5 | — |
| (−) | S. typhimurium | 20 | 20 | — | 17 | 20 | — | 13 | 18 | — | 11 | 13 | — | — | 11.5 | — |
| | S. enteritidis | 20 | 20 | — | 16.5 | 18 | — | 13 | 16 | — | 11 | 12 | — | — | 11 | — |
| | S. flexneri | 19 | 20 | — | 18 | 19 | — | 13 | 16 | — | 10.5 | 12 | — | — | 10.5 | — |
| | S. sonnei | 19 | 20 | — | 17 | 19 | — | 12 | 15 | — | 11 | 12 | — | — | — | — |
| | V. parahaemolyticus | 19 | 20 | — | 18 | 20 | — | 14 | 17 | — | 13 | 14 | — | 11 | 11.5 | — |
| | B. bronchiseptica | 20 | 20 | — | 18 | 18 | — | 14 | 14 | — | 12 | 12 | — | — | — | — |
| | Y. enterocolitica | 19 | 20 | — | 17 | 19 | — | 13 | 15 | — | 11.5 | 13 | — | — | 10.5 | — |
| | L. pneumophila | 18 | 20 | — | 16 | 18 | — | 12 | 14 | — | 11 | 12.5 | — | — | — | — |
| | A. salmonicida | 19 | 20 | — | 17 | 18 | — | 13 | 15 | — | 12 | 13 | — | 11 | 10.5 | — |
| | N. meningitidis | 18 | 20 | — | 16 | 18 | — | 12 | 15 | — | 11 | 13 | — | — | 10.5 | — |
| | M. catarrhalis | 18 | 20 | — | 15 | 17 | — | 11 | 13 | — | 11 | 12 | — | — | — | — |
| | A. ureae | 19 | 20 | — | 17 | 18 | — | 12 | 14 | — | 11 | 12 | — | — | — | — |

*A: S-(−)-tulipalin B, B: acetylated-S-(−)-tulipalin B, and C: R-(+)-tulipalin B

### Experimental Example 2

### Antibacterial effects of S-(−)-tulipalin B and acetylated-S-(−)-tulipalin B on Pathogenic Bacteria (Anaerobic)

In order to investigate antibacterial effects of S-(−)-tulipalin B, acetylated-S-(−)-tulipalin B and R-(+)-tulipalin B on pathogenic bacteria (anaerobic), antibacterial activity of these compounds was tested for 8 anaerobic bacterial species (Gram-positive spp: Propionibacterium acne ATCC 6919, Propionibacterium granulosum ATCC 25564, Clostridium perfringens KCCM 12098, Clostridium dificile KCCM 12115, and Corynebacterium glutamicum KCTC 1445; and 3 Gram-negative spp: Campylobacter jejuni KCTC 5327, Helicobacter pylori KCTC 5335, and Bacteroides fragilis KCTC 3688) in the same manner as in Experimental Example 1. The results thus obtained are given in Table 3 below. As can be seen from the results of Table 3, both of S-(−)-tulipalin B and acetylated-S-(−)-tulipalin B exhibited relatively high antibacterial effects on 8 anaerobic bacterial spp, whereas R-(+)-tulipalin B exhibited no antibacterial activity even at a concentration of 1.0%.

TABLE 3
Antibacterial effects of tulipalin B on anaerobic bacteria

| | | Treatment concentration (%)/Inhibitory zone diameter | | | | | | | | | | | | | | |
|---|---|---|---|---|---|---|---|---|---|---|---|---|---|---|---|---|
| | | (mm) | | | | | | | | | | | | | | |
| | | 1.0 | | | 0.3 | | | 0.1 | | | 0.03 | | | 0.01 | | |
| | | A* | B | C | A | B | C | A | B | C | A | B | C | A | B | C |
| Gram (+) | P. acne | 16 | 18 | — | 14 | 16 | — | 13 | 14 | — | 11 | 12 | — | — | 11 | — |
| | P. granulosum | 16 | 16 | — | 15 | 14 | — | 13 | 14 | — | 10.5 | 11.5 | — | — | 10.5 | — |
| | C. perfringens | 14 | 15 | — | 13 | 14 | — | 11 | 12 | — | — | 11 | — | — | — | — |
| | C. difficile | 13 | 15 | — | 13 | 13 | — | 10.5 | 11 | — | — | — | — | — | — | — |
| | C. glutamicum | 12 | 12 | — | 11 | 12 | — | 10.5 | 11 | — | — | — | — | — | — | — |
| Gram (−) | C. jejuni | 18 | 20 | — | 16 | 18 | — | 15 | 17 | — | 13 | 14 | — | 11.5 | 12 | — |
| | H. pylori | 18 | 20 | — | 16 | 17 | — | 14 | 15 | — | 12 | 13 | — | 10.5 | 12 | — |
| | B. fragilis | 18 | 20 | — | 16 | 16 | — | 14 | 15 | — | 12 | 13 | — | 10.5 | 11 | — |

*A: S-(−)-tulipalin B, B: acetylated-S-(−)-tulipalin B, and C: R-(+)-tulipalin B

### Experimental Example 3

### Minimum Inhibitory Concentration (MIC) of S-(−)-tulipalin B and acetylated-S-(−)-tulipalin B

The growth inhibitory activity of tulipalin B on each bacterial spp. was assayed by determining the minimum inhibitory concentration (MIC) of S-(−)-tulipalin B and acetylated-S-(−)-tulipalin B on the above-exemplified test bacteria according to a broth dilution method. That is, each 2 mL of the test bacteria, which were diluted to a concentration of 2×10⁶ CFU/mL, was added to 5 mL tubes. Then, each 50 μl of S-(−)-tulipalin B and acetylated-S-(−)-tulipalin B solutions, which were diluted to a concentration of 0.01 to 20 mg/mL, was added to the above individual tubes. Then, the bacteria were cultured at 37° C. for 24 hours, and MIC was defined as a minimum concentration that exhibits 100% inhibition of apparent bacterial growth. The results thus obtained are given in Table 4 below. As can be confirmed from Table 4, MIC of S-(−)-tulipalin B on aerobic bacteria exhibited a value of 0.01% to 0.03% for Gram-negative spp. and a value of 0.04% to 0.11% for Gram-positive spp., respectively. Further, acetylated-S-(−)-tulipalin B exhibited MIC of 0.005% to 0.01% for Gram-negative spp., and MIC of 0.03% to 0.08% for Gram-positive spp., respectively. Particularly, it was confirmed that both of S-(−)-tulipalin B and acetylated-S-(−)-tulipalin B exhibit significantly higher antibacterial activity on the Gram-negative group of bacteria, as compared to the Gram-positive group of bacteria.

On the other hand, MIC of S-(−)-tulipalin B on anaerobic bacterial exhibited a value of 0.03% to 0.1% for Gram-positive spp. and a value of 0.008% to 0.01% for Gram-negative spp. In addition, acetylated-S-(−)-tulipalin B exhibited MIC of 0.02% to 0.08% for Gram-positive spp. and MIC of 0.005% to 0.08% for Gram-negative spp., respectively. Similar to the results shown in aerobic bacteria, it was confirmed that both of S-(−)-tulipalin B and acetylated-S-(−)-tulipalin B exhibit higher antibacterial activity on Gram-negative anaerobes, as compared to Gram-positive anaerobes.

TABLE 4
MIC of S-(−)-tulipalin B

MIC (%)

| Bacterial strains | | | A* | B |
|---|---|---|---|---|
| Aerobic | Gram (+) | S. mutans | 0.10 | 0.03 |
| bacteria | | S. aureus | 0.08 | 0.03 |
| | | P. aeruginosa | 0.11 | 0.08 |
| | | L. monocytogenes | 0.10 | 0.03 |
| | | B. subtilis | 0.04 | 0.03 |
| | Gram (−) | E. coli | 0.01 | 0.005 |
| | | S. typhimurium | 0.03 | 0.008 |
| | | S. enteritidis | 0.03 | 0.008 |
| | | S. flexneri | 0.025 | 0.01 |
| | | S. sonnei | 0.03 | 0.02 |
| | | V. parahaemolyticus | 0.02 | 0.005 |
| | | B. bronchiseptica | 0.03 | 0.02 |
| | | Y. enterocolitica | 0.015 | 0008 |
| | | L. pneumophila | 0.03 | 0.01 |
| | | A. salmonicida | 0.01 | 0.008 |
| | | N. meningitidis | 0.01 | 0.008 |
| | | M. catarrhalis | 0.03 | 0.01 |
| | | A. ureae | 0.03 | 0.01 |
| Anaerobic | Gram (+) | P. acne | 0.03 | 0.02 |
| bacteria | | P. granulosum | 0.03 | 0.03 |
| | | C. perfringens | 0.08 | 0.03 |
| | | C. difficile | 0.10 | 0.03 |
| | | C. glutamicum | 0.10 | 0.08 |
| | Gram (−) | C. jejuni | 0.008 | 0.005 |
| | | H. pylori | 0.01 | 0.005 |
| | | B. fragilis | 0.01 | 0.08 |

*A: S-(−)-tulipalin B, and B: acetylated-S-(−)-tulipalin B

### Comparative Experimental Example 1

Analogously to Experimental Example 1, antibacterial activity of conventional natural (DF-100) and synthetic (Germall II and sorbic acid) antibacterial agents was investigated for 5 species of representative food-poisoning bacteria. The results thus obtained are given in Table 5 below.

TABLE 5

| | MIC (%) | | | |
|---|---|---|---|---|
| | S-(−)-tulipalin | | | |
| Bacterial strains | B | DF-100 | Germall II | Sorbic acid |
| S. aureus | 0.08 | 0.12 | 0.03 | 0.04 |
| E. coli | 0.01 | 0.05 | 0.005 | 0.01 |
| S. typhimurium | 0.03 | 0.07 | 0.02 | 0.03 |
| S. enteritidis | 0.03 | 0.06 | 0.01 | 0.01 |
| V. parahaemolyticus | 0.02 | 0.05 | 0.02 | 0.03 |

As can be seen from the results of Table 5, S-(−)-tulipalin B which is a natural antibacterial agent in accordance with the present invention exhibited antibacterial activity comparable to that of conventional synthetic antibacterial agents, but it was confirmed that S-(−)-tulipalin B exhibits superior antibacterial activity to DF-100 which is most prevalently used as a natural antibacterial agent.

### Experimental Example 4

### Non-Cytotoxicity of S-(−)-tulipalin

The cytotoxicity of S-(−)-tulipalin B was tested on HaCaT cells (human keratinocyte cell line) and normal human fibroblasts, according to an MTT assay. The results thus obtained are given in Table 6 below. Substantially no cytotoxicity was detected at concentrations less than 5% of S-(−)-tulipalin B, while slight cytotoxicity was observed when S-(−)-tulipalin B was added at a concentration higher than 5%. Upon considering the fact that no cytotoxicity was observed at a concentration of less than 5% at which antibacterial activity of S-(−)-tulipalin B was substantially exerted, it is believed that S-(−)-tulipalin B of the present invention can be safely used in cosmetics and pharmaceuticals.

TABLE 6
Cytotoxicity of S-(−)-tulipalin

| | Concentration of S-(−)-tulipalin B (%) | | | | | | | |
|---|---|---|---|---|---|---|---|---|
| | 0 | 0.13 | 0.25 | 0.50 | 1.00 | 2.00 | 5.00 | 8.00 |
| Cell viability | 100 | 100 | 101 | 99.9 | 99.7 | 99.5 | 97.2 | 88.6 |
| (%) | | | | | | | | |

### Experimental Example 5

### Thermal Stability of S-(−)-tulipalin B

In order to examine thermal stability of the title compound, S-(−)-tulipalin B was heat-treated at various temperatures of 40, 80, 100 and 120° C. for 1 hour. Analogously to Experimental Example 1, antibacterial activity of S-(−)-tulipalin B on E. coli was measured at a concentration of 0.02% according to a broth dilution method. The results thus obtained are given in Table 7 below.

TABLE 7
MIC of S-(−)-tulipalin B after heat treatment

| | Treatment Temp./MIC (%) | | | | |
|---|---|---|---|---|---|
| | Non- | | | | |
| Bacterial strains | treated | 40° C. | 80° C. | 100° C. | 120° C. |
| Escherichia coli | 0.01 | 0.01 | 0.01 | 0.01 | 0.015 |
| Staphylococcus aureus | 0.08 | 0.08 | 0.08 | 0.10 | 0.10 |
| Salmonella | 0.03 | 0.03 | 0.03 | 0.03 | 0.035 |
| typhimurium | | | | | |
| Salmonella enteritidis | 0.03 | 0.03 | 0.03 | 0.03 | 0.035 |
| Vibrio | 0.02 | 0.02 | 0.02 | 0.02 | 0.025 |
| parahaemolyticus | | | | | |

As can be seen from the results of Table 7, it was confirmed that S-(−)-tulipalin B exhibits substantially no difference in antibacterial activity between the heat-treated group and the control group (non-treated). Particularly from the fact that S-(−)-tulipalin B retains antibacterial activity even after heat treatment at 120° C., it can be seen that S-(−)-tulipalin B is very thermally stable. Therefore, it is believed that the antibacterial activity of S-(−)-tulipalin B is not thermally inactivated in view of heat processability.

### Experimental Example 6

### pH Stability of S-(−)-tulipalin B

In order to examine pH stability of S-(−)-tulipalin B, solvents were adjusted to pH of 2, 5, 7, 9, and 11 and a test sample was added thereto. The resulting solutions were allowed to stand at 37° C. for 1 hour and neutralized to a pH value of 7. Then, antibacterial activity of S-(−)-tulipalin B was measured in the same manner as in Experimental Example 1. The results thus obtained are given in Table 8 below.

TABLE 8
MIC of S-(−)-tulipalin B in response to pH changes

| | MIC (%) | | | | | |
|---|---|---|---|---|---|---|
| Bacterial strains | Non-treated | pH 2.0 | pH 5.0 | pH 7.0 | pH 9.0 | pH 11.0 |
| Escherichia coli | 0.01 | 0.015 | 0.01 | 0.01 | 0.01 | 0.015 |
| Staphylococcus aureus | 0.08 | 0.085 | 0.08 | 0.08 | 0.08 | 0.10 |
| Salmonella | 0.03 | 0.035 | 0.03 | 0.03 | 0.03 | 0.035 |
| typhimurium | | | | | | |
| Salmonella enteritidis | 0.03 | 0.035 | 0.03 | 0.03 | 0.03 | 0.035 |
| Vibrio | 0.02 | 0.025 | 0.02 | 0.02 | 0.02 | 0.025 |
| parahaemolyticus | | | | | | |

A significance test for antibacterial effects of S-(−)-tulipalin B was conducted in the same manner as in Experimental Example 1.

From no significant difference in antibacterial activity of S-(−)-tulipalin B under all the pH conditions when compared with the control group (non-treated), it can be seen that S-(−)-tulipalin B has excellent pH stability. Usually, bacteria exhibit optimum growth at around pH 6 to 7. Therefore, it is possible to inhibit bacterial growth by adjusting pH of growth surroundings to a low value. Most of antibacterial materials added to foods are acidulants. A typical one is vinegar. As can be seen from the results of Table 8, S-(−)-tulipalin B has a broad acceptable pH spectrum. Consequently, S-(−)-tulipalin B is expected to have diverse applicability as it not only has a different action mechanism than conventional synthetic preservatives, but also exhibits advantages of no deterioration in taste and characters of foods.

### Experimental Example 7

### Preservative Effects of S-(−)-tulipalin B in Cosmetics

Using a lotion containing 1% S-(−)-tulipalin B which was prepared in Example 1, preservative effects of S-(−)-tulipalin B were evaluated. The preservative effects were assayed according to the Microbial Challenge Test proposed by the European Pharmacopoeia Commission (E.P.) (1996) concerning topical preparations (Letters in Applied Microbiology 2002, 35, 385-389). This test was carried out on Escherichia coli, Pseudomonas aeruginosa and Staphylococcus aureus. The preservative effects of the test compound were measured four times at regular intervals of 5 days for 20 days. The results thus obtained are given in Table 9 below.

TABLE 9

| | Day 0 | Day 5 | Day 10 | Day 15 | Day 20 |
|---|---|---|---|---|---|
| | (Colony | (Colony | (Colony | (Colony | (Colony |
| Bacterial strains | counts/disc) | counts/disc) | counts/disc) | counts/disc) | counts/disc) |
| Escherichia coli | 1.5 × 10⁴ | 2 | 0 | 0 | 0 |
| Pseudomonas | 1 × 10⁴ | 2 | 0 | 0 | 0 |
| aeruginosa | | | | | |
| Staphylococcus | 2 × 10⁴ | 8 | 0 | 0 | 0 |
| aureus | | | | | |

As shown in Table 9, when a lotion containing 1% S-(−)-tulipalin B was subjected to a preservative test for 20 days, almost all microorganisms were killed within 10 days after bacteria were treated with S-(−)-tulipalin B. This result demonstrates that S-(−)-tulipalin B of the present invention has potent preservative activity on a variety of bacteria.

### Experimental Example 8

### Safety of S-(−)-tulipalin B for Human Skin

In order to confirm whether S-(−)-tulipalin B, which was identified to have excellent antibacterial and preservative activity as above, is safe on human skin, a skin safety evaluation was carried out. The evaluation was made according to a skin cumulative irritation test.

Squalene-based formulations containing 0.1%, 1%, 5%, and 10% of S-(−)-tulipalin B were applied to the forearm of 30 healthy adults through patches for 24 hours every other day, and the patches were replaced with fresh ones 9 times in total from after the first application in order to examine whether S-(−)-tulipalin B irritated the skin or not.

The patch test was performed using a Finn chamber (Epitest Ltd., Finland). The topical skin formulations were loaded dropwise in an amount of 15 μl per patch in the chamber. Every round of the patch application, the response of skin was scored using the following Equation 1. The results thus obtained are given in Table 10 below.

Avg. Response Degree=[[Response Index×Response Degree/Total No. of subjects×highest score(4 points)]×100]/No. of Test(9 rounds) Equation 1

In regard to the response degree, scoring was made as follows: ±=1, +=2, and ++=4. When the average response degree was less than 3, the composition was regarded as being safe for skin.

TABLE 10

| | Nos. of Subjects in response to test materials | | | | | | | | | | | | | | | | | | | | | | | | | | | Avg. |
|---|---|---|---|---|---|---|---|---|---|---|---|---|---|---|---|---|---|---|---|---|---|---|---|---|---|---|---|---|
| | Week 1 | | | | | | | | | Week 2 | | | | | | | | | Week 3 | | | | | | | | | Re- |
| Test | 1^{st} | | | 2^{nd} | | | 3^{rd} | | | 4^{th} | | | 5^{th} | | | 6^{th} | | | 7^{th} | | | 8^{th} | | | 9^{th} | | | sponse |
| materials | ± | + | ++ | ± | + | ++ | ± | + | ++ | ± | + | ++ | ± | + | ++ | ± | + | ++ | ± | + | ++ | ± | + | ++ | ± | + | ++ | Degree |
| Control | 2 | — | — | — | — | — | — | — | — | — | — | — | — | — | — | — | — | — | — | — | — | — | — | — | — | — | — | 0.18 |
| (Squalene) | | | | | | | | | | | | | | | | | | | | | | | | | | | | |
| (Group 1) | 0 | — | — | 0 | — | — | — | — | — | — | — | — | — | — | — | — | — | — | — | — | — | — | — | — | — | — | — | 0.00 |
| S-(−)- | | | | | | | | | | | | | | | | | | | | | | | | | | | | |
| tulipalin B | | | | | | | | | | | | | | | | | | | | | | | | | | | | |
| (0.1%) | | | | | | | | | | | | | | | | | | | | | | | | | | | | |
| (Group 2) | 0 | — | — | 0 | — | — | — | — | — | — | — | — | — | — | — | — | — | — | — | — | — | — | — | — | — | — | — | 0.00 |
| S-(−)- | | | | | | | | | | | | | | | | | | | | | | | | | | | | |
| tulipalin B | | | | | | | | | | | | | | | | | | | | | | | | | | | | |
| (1%) | | | | | | | | | | | | | | | | | | | | | | | | | | | | |
| (Group 3) | 1 | — | — | 0 | — | — | — | — | — | — | — | — | — | — | — | — | — | — | — | — | — | — | — | — | — | — | — | 0.09 |
| S-(−)- | | | | | | | | | | | | | | | | | | | | | | | | | | | | |
| tulipalin B | | | | | | | | | | | | | | | | | | | | | | | | | | | | |
| (5%) | | | | | | | | | | | | | | | | | | | | | | | | | | | | |
| (Group 4) | 2 | — | — | 1 | — | — | — | — | — | — | — | — | — | — | — | — | — | — | — | — | — | — | — | — | — | — | — | 0.27 |
| S-(−)- | | | | | | | | | | | | | | | | | | | | | | | | | | | | |
| tulipalin B | | | | | | | | | | | | | | | | | | | | | | | | | | | | |
| (10%) | | | | | | | | | | | | | | | | | | | | | | | | | | | | |

| | | | | | | | | | |
|---|---|---|---|---|---|---|---|---|---|
| No. of | 30 | 30 | 30 | 30 | 30 | 30 | 30 | 30 | 30 |
| subjects | | | | | | | | | |

As shown in Table 10, the subjects corresponding to ±, + and ++ in Test Group 3 numbered 1, 0 and 0, respectively, while the other groups showed no response. According to Equation 1, the average response degrees of Test Groups 1 to 4 were calculated to be 0.00, 0.00, 0.09 and 0.27, respectively, which are all less than 3, thus demonstrating that S-(−)-tulipalin B causes no noticeable cumulative irritation potential and is safe for human skin.

### Experimental Example 9

### Acute Oral Toxicity Test

In order to examine whether S-(−)-tulipalin B is safely applicable to foods, an acute oral toxicity test was carried out. For this purpose, 5 or 6-week old SPF SD rats (n=20) were purchased as laboratory animals and were orally administered with S-(−)-tulipalin B under the following breeding conditions.
Temperature and humidity: 22±2° C., RH 50±10%
Light/Dark (L/D) cycle: fluorescent lighting (light from 08:00 a.m. to 20:00 p.m.)
Illuminance: 200 to 300 Lux
All animals were allowed ad libitium access to UV-treated water
Dilutions of the test material in sterile distilled water were administered to animals, whereas the same volume of sterile distilled water was used as a control.

On the day of administration, the rats were observed for general conditions every hour for 4 hours after administration of a test drug. From 1 to 14 days after the administration, the rats were carefully examined once a day for general state, toxic symptoms, motility, appearance, changes in the autonomic nervous system, and death of animals. A variety of pathoanatomical investigations were also performed to determine the toxicity of S-(−)-tulipalin B. S-(−)-tulipalin B was determined to have LD₅₀ of 24500 mg/kg B.W. and no toxicity.

### EFFECT OF THE INVENTION

As apparent from the above description, tulipalin B of the present invention has extraordinary thermal stability and pH stability, potent antibacterial activity against a broad spectrum of microorganisms, and no toxicity or irritation in human safety tests. Therefore, the active compound or composition of the present invention is applicable to foods, cosmetics and pharmaceuticals, as a natural antibacterial agent.

What is claimed is:

1. An antibacterial composition comprising S-(−)-tulipalin B or acetylated-S-(−)-tulipalin B of formula 1 as an active ingredient: 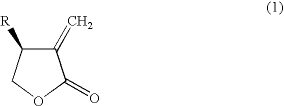
wherein R is hydroxy (—OH) or acetoxy (—OCOCH₃).

2. The composition according to claim 1, wherein the active ingredient is present in the range of about 0.001% to about 30% by weight, based on the total weight of the composition.

3. The composition according to claim 1, which is effective in inhibiting at least one bacteria selected from the group consisting of Streptococcus spp.; Salmonella spp.; Escherichia spp.; Staphylococcus spp.; Shigella spp.; Pseudomonas spp.; Listeria spp.; Vibrio spp.; Bacillus spp.; Propionibacterium spp.; Campylobacter spp.; Helicobacter spp.; Clostridia spp.; Bordetella spp.; Yersinia spp.; Legionella spp.; Aeromonas spp.; Bacteroides spp.; Neisseria spp.; Moraxella spp.; Corynebacterium spp.; and Actinobacillus spp.

4. The composition according to claim 1, wherein the active ingredient acts is a preservative.

5. The composition according to claim 1, wherein the composition comprises an antioxidant.

6. The composition according to claim 5, wherein the antioxidant is butylated hydroxytoluene (BHT).

7. The composition according to claim 6, wherein a content of BHT is about 0.001 ppm.

8. The composition according to claim 1, which is suitable for use in a pharmaceutical composition.

9. The composition according to claim 1, which is suitable for use in a cosmetic composition.

10. The composition according to claim 1, which is suitable for use in a food composition.

10. The composition according to claim 1, which is suitable for use in a fiber composition.
